(19) United States
(12) Patent Application Publication
(10) Pub. No.: US 2025/0279180 A1
(43) Pub. Date: Sep. 4, 2025

(54) BREASTMILK MANAGEMENT SYSTEM

(71) Applicant: MEDELA HOLDING AG, Baar (CH)

(72) Inventors: David Y. Cho, Arlington Heights, IL (US); Andy Giles, Crystal Lake, IL (US)

(21) Appl. No.: 18/591,262

(22) Filed: Feb. 29, 2024

(51) Int. Cl. G16H 20/60; A61J 15/00

(52) U.S. Cl. CPC G16H 20/60; A61J 15/0011

(57) ABSTRACT

A system and method for storing, fortifying, tracking, and administering breastmilk to an infant in a neonatal intensive care unit in an automated or semi-automated manner that minimizes manual tasks of NICU caregivers and provides interactive information to guide NICU caregivers though fortification and administration of breastmilk to the intended infant in a manner consistent with feed orders.

### FIELD

This disclosure relates generally to providing nourishment to infants in a neonatal intensive care unit.

### BACKGROUND

Neonatal intensive care units, or “NICU”'s, are where many premature infants, multiple-birth (twins, triplets, quadruplets, etc.) babies, and other newborns facing early health challenges, spend their first days, weeks, or in some cases months, while gaining strength, growing, and being provided around-the-clock medical attention and nutrition until they demonstrate the ability to thrive and can be released to home environments.

NICU patients, and especially very low birth weight (VLBW) and extremely low birth weight (ELBW) infants, are particularly susceptible to necrotizing enterocolitis (NEC). Studies show a mother's breastmilk is effective in mitigating risks of NEC. For example, Parker, M.D., et al., “Promoting Human Milk and Breastfeeding for the Very Low Birth Weight Infant,” AMER. ACAD. OF PEDIATRICS, Vol. 148, No. 5 (November 2021); Meek, M.D., et al., “Technical report: Breastfeeding and the Use of Human Milk,” AMER. ACAD. OF PEDIATRICS, Vol. 150, No. 1 (June 2022); and Meier, PhD, RN, FAAN, et al., 16 “Human Milk in the Neonatal Intensive Care Unit,” in Breastfeeding and Breastmilk—from Biochemistry to Impact—A Multidisciplinary Introduction (Ed., Family Larsson-Rosenquist Foundation) Georg Theime Verlag K G, The Global Health Network (Jul. 1, 2018). The beneficial flora provided by a mother's breastmilk to her own baby, as a continued source of maternal mitochondrial DNA (mtDNA) that was being provided in utero, is helpful to aid in the development of the small intestines, the cellular interstices of which are not even fully developed at full-term birth. See generally Cosemans, et al., “Breastfeeding Predicts Blood Mitochondrial DNA Content in Adolescents,” Scientific Reports, Vol. 10, 387 (2020).

In most NICU's, the infant is separated from its mother, which presents a practical impediment to providing breastmilk from the baby's mother. Depending on the duration of stay in the NICU, nursing mothers are often discharged from the hospital well before their babies are released, thus imposing additional challenges of collecting, transporting, identifying, and storing the mother's breastmilk. The mother's milk must be expressed, and ideally, collected, stored, and maintained, safely and with some frequency. Also, the infant(s) will need to be fed periodically, such as every 3 or 4 hours.

NICU professionals are tasked with a number of duties during their shifts. Among these are the tedious, but important, tasks of matching collected breastmilk to the infant whose mother provided the breastmilk, fortifying the collected breastmilk to the specifications of the neonatologist, pediatrician, or other attending physician (which may include mixing breastmilk with additives to enhance the carbohydrate, protein, and fat content of the mother's breastmilk, and, in some cases, subsidizing or combining the mother's breastmilk with donor milk and/or formula), monitoring stored breastmilk to ensure it is maintained within acceptable temperature ranges, warming the breastmilk to proper feeding temperature, feeding the infant, determining and charting the volume of breastmilk consumed by the infant, among many other tasks indirectly related, or unrelated, to feeding, such as diaper changes, monitoring vital signs, taking blood samples, and administering medications.

Even in well-staffed NICU's, where the nurse-to-patient ratio is as favorable as 1:2, the chores relating to feeding a mother's breastmilk to her infant occupy a disproportionate amount of caregiver time. Existing breastmilk fortification techniques require the caregiver to mix a number of powdered or liquid fortification materials in specific quantities and blend the fortification mixture with the breastmilk to be fortified. This introduces opportunity for human error, and even with great precision in preparing the mixture of ingredients, it is difficult to ensure homogeneous distribution of the fortified materials in human breastmilk. When NICU patients, particularly those fed by mouth, consume less than the preferred or prescribed volume of breastmilk, as they often do, it is difficult to determine with certainty the content of fortifiers in the portion of the breastmilk the infant consumed.

Various attempts have been made to address some of the above challenges. For instance, U.S. Pat. No. 8,775,209, assigned to Haemonetics Corporation, entitled Apparatus and Method for Administration of Mother's Milk, disclosed a method for matching of mother's breastmilk to the correct baby in a hospital environment where the mother's breastmilk is expressed in advance and stored for later feeding. The disclosed methods involve providing electronically readable indicia to confirm the baby's identity and correlate the mother's breastmilk to the appropriate baby. The disclosure additionally described methods and apparatus for collecting, storing, and communicating data regarding the handling of the breastmilk so as to record audit trails and provide caregiver guidance.

U.S. Pat. No. 8,172,129, assigned to Paragon Data Systems, entitled Method for Tracking Breast Milk in a Neonatal Care Facility, disclosed the use of optically-scannable identifiers, such as barcodes, affixed to breastmilk containers and measuring devices to ensure correlation of a mother's breastmilk and her baby.

However, the number of steps the NICU practitioners must perform in the exercise of the methods discussed in these approaches, coupled with the additional array of tasks that must be performed in a single shift to provide nourishment and care to each infant in the NICU, are not adequately addressed by these disclosures.

### SUMMARY

A comprehensive breastmilk collection, identification, identity verification, fortification, storage, and delivery system of the present disclosure consolidates, automates at least various aspects of, and simplifies, breastmilk handling tasks that, using conventional approaches, occupy an inordinate amount of time for NICU caregivers. Through automation or semi-automation, the number of physical interaction with each given supply of breastmilk can be significantly reduced.

A system for NICU breastmilk management can best be described with respect to three main phases: A milk collection phase, a milk preparation phase, and a milk delivery phase. In the milk collection phase, a nursing mother collects expressed breastmilk, typically at home, in a collection container. The collection container may be intended for single-use, i.e., be disposable, or may be reusable, in which case the collection container is washable and able to be sanitized.

The collection container is provided with identifying indicia, which may be in a machine readable format and/or may be visually readable. The identifying indicia may include such information as the identity of the mother, the identity of the infant for whom the milk is intended, the date of birth of the infant, the volume of milk collected in the container, the date and time of the collection, as well as the start and end times of, and volume of milk collected during, one or more pumping sessions.

The information associated with the breastmilk collected in the collection container, generally referred to herein as identification indicia associated with the collection container, can be stored on a non-transitory computer readable medium such as in a smartphone memory or at an external receiver. The external receiver may be a cloud-based electronic medical record system hosted by the hospital, with a patient record associated with the infant, the mother, or both. Alternately, the external receiver may be hosted by a third party. Alternatively or additionally, the identification indicia can be stored on a computer chip or other non-transitory data storage media carried on or in the collection container itself, or written in a machine-readable and/or human-readable format, such as in a printed QR-code, bar code, and/or text secured to the collection container.

Having the capacity to remotely communicate with the non-transitory computer readable medium on which information associated with the breastmilk contained in the collection container permits the NICU where the infant is staying to access and receive data concerning in-bound breastmilk intended for the infant even before that particular supply of breastmilk reaches the NICU. Advanced notice can provide the caregiver with helpful information to prepare for the arrival of the milk supply, such as whether any donor milk may be necessary, and if so, how much, and what fortifiers might be needed to mix with the milk prior to feeding the infant. Other information the caregiver might learn about inbound milk supply includes whether the breastmilk has exceeded its expiration time and date, the extent to which it may require temperature adjustment through refrigeration, and, if the NICU already has prior collected breastmilk intended for a given infant, the NICU will be able to more effectively prioritize the use of the inbound and stored breastmilk supply containers.

The NICU may even be able to communicate the status of its supply to the mother, and depending on the mother's distance to the NICU, this may enable the mother to plan her pumping sessions and trips (either by the mother or someone else on the mother's behalf) to the NICU with further breastmilk collection containers. For instance, the NICU may be able to communicate with the mother via the same communication system, or through a different patient portal, to provide the mother information as to a quantity of milk the mother is encouraged to bring to the hospital, and discourage the mother from bringing milk in excess of the recommended quantity to the NICU. Communication to a NICU of the quantity of milk expected to arrive at the NICU may alternatively be useful as part of a donor milk program, where, with appropriate consents, milk delivered to the NICU in excess of a quantity recommended for a particular mother's infant in the NICU could be made available as donor milk to other infants in the NICU whose mothers deliver less milk than is recommended for their babies, or to provide donor milk having a composition that is complementary to that of the milk from a mother with which it is mixed, which may include donor milk of like composition, or alternatively, donor milk of a different composition that is intended to fortify the milk with which it is mixed to bring the resulting mixture closer to a milk composition recommended by a neonatologist or other clinician.

During a storage phase, the breastmilk collection containers may be stored directly in a storage area within a NICU. The storage area may be a refrigerator for breastmilk to be fed to a baby within 3 to 4 days. A freezer may be used as the storage area for breastmilk to be stored for longer periods of time. However, once unfrozen, the breastmilk collection container is preferably provided with data indicative of a time and date the breastmilk was unfrozen, and an indication to the caregiver that the contents of the present collection container is to be fortified as-needed and fed to the infant, or utilized as donor milk, within 24 hours of being unfrozen.

Because of the identifying information associated with the identifying indicia provided on or in association with a given breastmilk collection container, the precise location of each breastmilk collection container within the storage area need not be carefully tracked, which was a requirement of many conventional NICU breastmilk handling systems to assure proper association of a particular mother's breastmilk to her infant(s) in the NICU.

During a milk preparation phase, automated procedures are employed prior to administration of breastmilk to an infant. A first step of the preparation involves determination of the milk mixture, and a second step involves actual mixing. An automated mixing system may be used to introduce to breastmilk in the breastmilk collection container a calculated quantity of one or more additives from one or more of a plurality of fortifiers. Alternatively, the fortification may be performed manually, in which case a fortification station, or feed system, is used to support the collection container, and a display associated with the fortification station displays instructions to the NICU caregiver as to an identification and quantity of one or more fortifiers to be added to the breastmilk in the collection container to achieve, or at least approach, the prescribed mixture. During a mixing step, the contents of the breastmilk collection container, including the mother's expressed breastmilk and the calculated contents of the one or more of the plurality of fortifiers, is mixed in an effort to obtain homogeneity.

A milk delivery phase involves, in the case of automated or semi-automated delivery, placing the contents of the breastmilk collection container into fluid communication with a delivery pump. As many NICU infants are fed through a nasogastric feeding tube, the delivery pump may be networked in a fluid circuit to output to the nasogastric feeding tube of the associated infant. As a confirmatory step, prior to initiation of milk delivery, the automated system could confirm that the identifying indicia associated with the breastmilk collection container is a match for the infant with which the nasogastric feeding tube to which the delivery pump is connected.

### BRIEF DESCRIPTION OF THE DRAWINGS

FIG. 1 is a semi-schematic diagram of a typical conventional workflow or sequence of receiving, fortifying, storing, and administering breastmilk in a neonatal intensive care unit (NICU);

FIG. 2 is a first semi-schematic diagram of a streamlined workflow in a NICU according to the systems and methods of the present disclosure; and

FIG. 3 is a second semi-schematic diagram of a streamlined workflow in a NICU according to the systems and methods of the present disclosure.

### DETAILED DESCRIPTION

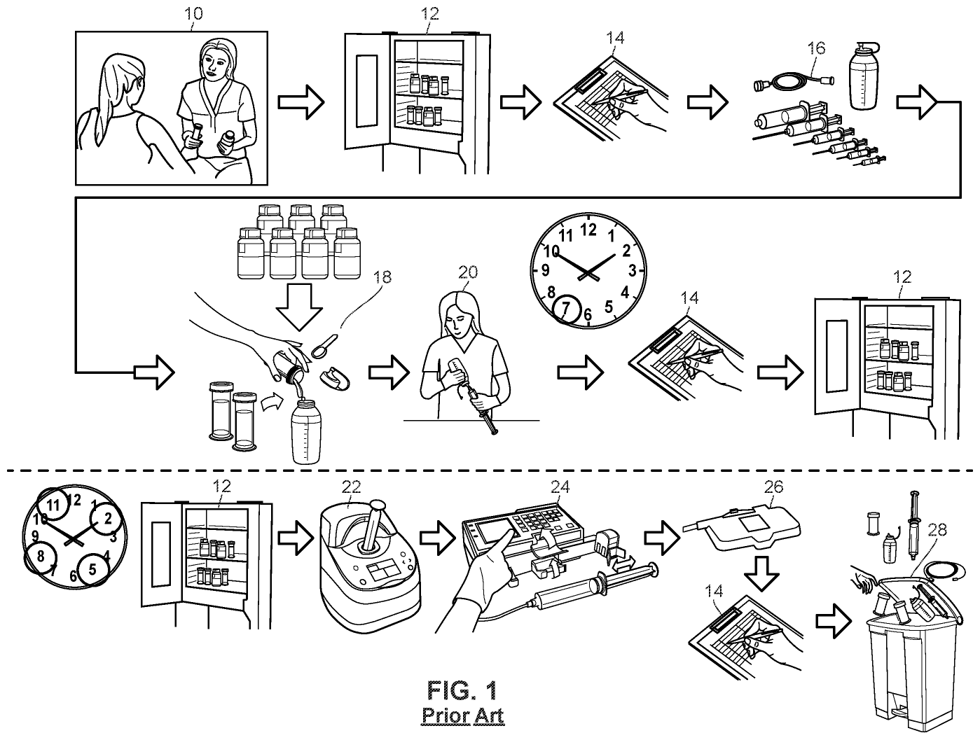

Each neonatal intensive care unit (NICU) develops its own specific preferred protocols and workflows for handling and administering breastmilk to its patients, most of whom are premature infants and some of whom are of very low birth weight (VLBW) or extremely low birth weight (ELBW). However, a common constraint on all NICU's is the number of labor-intensive steps involved in storing, maintaining, fortifying, and delivering the correct breastmilk, with the desired concentration of fat, protein, and carbohydrates to the infants in their care. Referring to FIG. 1, a workflow for breastmilk management within a NICU may include such steps as receiving breastmilk from (or on behalf of) an infant patient's mother at a receiving station 10, storing and securing the received breastmilk in a temperature-controlled storage unit 12, recording information pertaining to the received breastmilk such as in an electronic medical record (EMR) or an analog chart 14, procuring and preparing sterile feeding supplies 16, fortifying the breastmilk to a pediatrician, neonatologist, or other attending physician or other caregiver's specifications at a fortification station 18, portioning the fortified breastmilk into a feeding system, such as an enteral feeding syringe, at a portioning station 20, performing additional recording of information relative to the fortified and portioned breastmilk in the EMR or analog chart 14, and storing unused breastmilk, both fortified and any yet-to-be fortified, back in the storage unit 12. These aforementioned steps must be performed at least once every twelve hours, or twice per day. Then, depending on each infant-patient's feeding schedule, which may be every three hours, or eight times per day per patient, additional steps that are typically performed include retrieving fortified breastmilk from the storage unit 12 a few minutes prior to feeding, warming fortified breastmilk for administration by placing it in a warming station 22, such as a breastmilk warming device as disclosed in U.S. Pat. Nos. 9,241,596 and 10,327,585 (which are incorporated herein by reference), further preparing fortified and warmed breastmilk for administration by, for example, placing a syringe containing the warmed, fortified breastmilk into a fluid administration pump 24, additionally warming the breastmilk with an in-line warming system 26, recording yet additional information concerning the administered breastmilk in the EMR or analog chart 14, and disposing of the used supplies in a suitable waste collection receptacle 28.

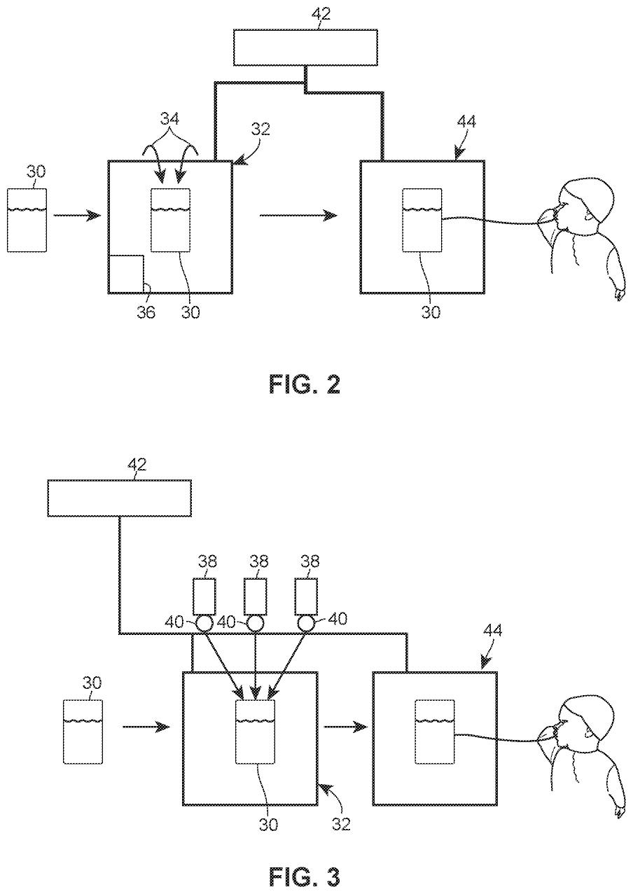

By employing the systems and methods of the present disclosure, the workflow in a NICU environment can be significantly reduced, with improved efficiency and reliability at most, if not all, stations, and greatly easing the burden on caregivers in a NICU. Comparing the flowchart of FIG. 1 to that of FIG. 2, it will be appreciated that a typical breastmilk management process employing the systems and methods of the present disclosure can reduce the steps from over a dozen to as few as eight or less.

The system according to the invention includes a collection container 30 where the mothers collect the breast milk after expression, usually at home, The collection containers 30 filled with milk can be stored at home and can then be brought to the hospital, e.g. once a day, every other day, whenever possible, or when required, as the case may be.

The collection containers 30 filled with milk are preferably maintained in a refrigerated environment, such as a fridge, both at home and in the hospital (but may also be frozen or stored in another way).

The system further includes a fortification station 32 for adding fortifiers 34 to the milk. The fortification station 32 may be a manual fortification station or an automatic fortification station.

In the first case (FIG. 2), the fortification station 32 has a support for the container and an output system 36, such as a display, for providing the healthcare professional with instructions of the fortification of the milk, as explained below.

In the latter case (FIG. 3), the fortification station 32 has reservoirs 38 for the fortifiers, each provided with a dispensing unit 40 that can be controlled (i.e. opened or closed) independently from the others. A computer system 42 is connected to and drives the dispensing units 40 for introducing the fortifier into the breastmilk.

The system further includes a pumping station 44 for delivering the breastmilk to the infant; the pumping station may e.g. be a traditional pumping station with a syringe pump, but other options are possible.

Preferably, the container where the milk is collected at home is used over the whole process, from milk collection at home to milk delivery to the infant and is thus also used at the fortification station and at the delivery station. Alternatively, different containers may be used at home and in the hospital, e.g. the milk collected at home in a first container is transferred into a second container when it is received at the hospital; at the hospital the milk may also be transferred again to a third container, so that e.g. at the fortification station and at the delivery station different containers are used; so one, two or more than two containers may be used. In addition, the container or containers are manually transferred to the fortification station and then to the delivery station. Automatic transfer is possible to reduce loss due, for example, to spillage during manual transfers.

The computer system 42 is connected to and drives the fortification station 32 and the delivery station 44. The computer system 42 may be implemented by the control unit of the fortification station 32 or the control unit of the delivery station 44 or may be implemented by the control unit of the fortification station 32 and delivery station 44 that are operatively connected to one another; the computer system 42 may also be an external computer system, possibly of general type such as a laptop or a tablet, or may also be a cloud based computer system. The computer system 42 is connected to the fortification station 32 and/or delivery station 44 by one or more wires or wirelessly.

The computer system 42 has a processing unit, a non-transitory computer readable medium, input devices such as a keyboard, scanner, mouse or others.

The processing unit is arranged for receiving first data indicative of the energy content and/or nutrient content and/or micro nutrient content of the provided breastmilk and for receiving second data indicative of the energy content and/or nutrient content and/or micro nutrient content of a feed order for the infant to whom the breastmilk is to be administered.

The first data and the second data may be stored in the non-transitory computer readable medium or one of the first and second data or both may be received via the input devices.

The processing unit can compare the first data with the second data and, if the first data do not match the second data, the processing unit can automatically determine at least one fortifier and the quantity thereof to be added to the breastmilk for matching the first data and the second data.

In the embodiment with a manual fortification station the computer system can display the determined fortifier and amount thereof on the display 36, so that the healthcare professional can manually add the fortifiers into the milk according to the instructions from the display 36.

In the embodiment with an automatic fortification station the computer system can drive the dispensing units 40 of each of the reservoirs 38 in order to introduce the required amount of the required fortifier into the milk.

In a method for administering breastmilk to an infant, first, collected breastmilk is provided. The first data indicative of the energy content and/or nutrient content and/or micro nutrient content of the provided breastmilk are provided; the energy content can be in required Kcal to be fed to the infant or nutrients like e.g. fat or protein or carbohydrates that have to be fed to the baby. Micronutrients, such as electrolytes and vitamins, could be provided as well.

Different ways for providing the first data are possible. The first data may come from average data available in scientific literature. Alternatively the first data may be determined on a case to case basis, e.g. by analyzing the data and determining the required energy and/or micro and/or macro nutrients thereof. Known methods can be used to such an end, like e.g. spectroscopy.

Infants in NICUs are fed according to feed orders. Feed orders are receipts or instructions provided by a neonatologist and indicate the feed to be given to the baby, in terms of e.g. caloric content and/or macronutrients (fat, carbohydrates, protein) and/or micronutrients. Infants shall be fed according to the feed order (and thus doctor's instructions).

Second data indicative of the energy content and/or nutrient content and/or micro nutrient content of a feed order for the infant to whom the breastmilk is to be administered are provided as well. For example the caloric content and the fat and protein amount are defined as second data.

First and second data can thus be compared in order to determine whether they match.

Matching of the first and second data does not necessarily requires that they are exactly the same. Clinical practice usually accepts a match when the first data over match the second data, i.e. the first data is larger than the second data.

In case the first data matches the second data, no additional action is required and the milk can be fed to the baby as is; though this is unlikely to occur, because infants in NICUs can typically only receive very limited amounts of feed, so that the breastmilk needs to be powered in terms of calories and/or macro nutrients contents and/or micro nutrients content as the case may be.

If the first data do not match the second data, the milk needs to be fortified, this meaning that fortifiers are added to the milk in order to increase its caloric content and/or macro nutrients content and/or micronutrient contents thereof. Advantageously the amount of fortifiers to be added to the milk is determined automatically, i.e. without any need for human intervention, apart from the need to provide the required information, such as second data.

In addition, both the fortifier, i.e. its type, and the amount of the fortifier, i.e. its quantity, to be added to the breastmilk to bring the first data closer to the second data, are automatically detected.

Once the fortifiers and the quantity thereof are determined, they can be introduced into the breastmilk.

While the determination phase is automatic, the present introduction phase may be automatic or not. Therefore once the fortifiers and the amount thereof have been determined, they can be manually added to the breastmilk. Alternatively, once the fortifier and the amount thereof have been automatically determined, they can be automatically added to the breastmilk. Automatically added to the breastmilk is to be intended such that it occurs without human intervention, human supervision may anyway be required.

The milk so fortified can thus be fed to the infant. Such as via a pump e.g. of known type such as syringe pumps, supplied with the fortified breastmilk that is then fed to the infant.

In the following an example of a way for determining the fortifier and the amount thereof is explained.

A database of fortifiers may be provided, the database including all fortifiers available for being added to the breastmilk.

The database includes a link for each fortifier in the database to the energy content and/or nutrient content and/or micro nutrient content; this way from the database it would be possible to extract an information on which fortifier is to be used when the caloric content, macro nutrients, micro nutrients are to be increased. The link may be, in different examples, the content of caloric content, macro nutrients, and/or micro nutrients, so that if the feed order requires e.g. a specific macro nutrient, the macro nutrient included in the database with the higher amount of the required macro nutrient is selected. Another possibility is to link each fortifier to a calorie content or to a specific macro nutrient or micro nutrient, so that when e.g. calorie content is required the fortifier linked to the calorie content is selected from the database.

This way, since from the comparison results the required energy content and/or nutrient content and/or micro nutrient that are needed to bring the second data closer to the first data, after the comparison it can be easily identified the specific fortifier that is to be added to the breastmilk. Therefore the fortifier linked to the energy content and/or nutrient content and/or micro nutrient content that corresponds to the required energy content and/or nutrient content and/or micro nutrient content can be selected as the one to be added to the breastmilk.

Likewise, in order to determine the quantity of the at least one fortifier, the database has also a link for each fortifier to a basic amount thereof; e.g. a specific fortifier may be available in servings having a predetermined caloric content and/or macro nutrient content and/or micro nutrient content.

From the comparison, the required amount of energy content and/or nutrient content and/or micro nutrient that are required to match the first data with the second data are determined, so that the number of basic amounts of the selected fortifier can be calculated that brings the energy content and/or nutrient content and/or micro nutrient content closest to the required energy content and/or nutrient content and/or micro nutrient content.

As an example, if the feed order requires 20 Kcal, 20 mg fat, and 20 mg proteins, from the database it is possible to extract information on the fortifiers that are linked to caloric content, fat and protein, e.g. fortifier A is linked to calorie content, fortifier B to fat and fortifier C to protein. From this data it is possible to calculate the amount required of fortifier A (required calories minus calories in fortifier B minus calories in fortifier C), the amount required of fortifier B (required fat minus fat in fortifier A minus fat in fortifier C), the amount required of fortifier C (required protein minus proteins in fortifier A minus proteins in fortifier B).

Preferably, providing breastmilk includes collecting breastmilk in a collection container; storing the collection container in a storage device such as a fridge; retrieving the collection container from the storage device when required. Preferably, in particular when the breastmilk is stored in a fridge but not only, the fortified breastmilk is heated before being delivered it to the infant, in order to bring it to a temperature acceptable for the infant. The milk may also be pasteurized.

The present invention also refers to a software product for running on the computer system. The software has instructions that, when read by a processing unit of the computer system, cause the processing unit to:
receive the first data indicative of the energy content and/or nutrient content and/or micro nutrient content of the provided breastmilk
receive the second data indicative of the energy content and/or nutrient content and/or micro nutrient content of a feed order
compare the first data with the second data and
automatically determine at least one fortifier and the quantity thereof to be added to the breastmilk for matching the first data and the second data, if the first data do not match the second data.

In particular the instructions for receiving first data and/or second data may cause the CPU to retrieve the first data and/or the second data from one or more database. The databases may be stored in the non-transitory computer readable medium of the computer system or may be stored on external devices accessible by the processing unit of the computer system. The processing unit may also retrieve the first data and/or second data from an input device, such as a keyboard or a scanner or other.

When the software has the databases, the databases can store the fortifiers and can link each fortifier to the energy content and/or nutrient content and/or micro nutrient content thereof; the software can thus include instructions for determining from the comparison the required energy content and/or nutrient content and/or micro nutrient that are required to match the first data with the second data and selecting from the database the fortifier linked to the energy content and/or nutrient content and/or micro nutrient content that corresponds to the required energy content and/or nutrient content and/or micro nutrient content.

In addition, the database can also link each fortifier to basic amounts thereof and the software can so have instructions for determining from the comparison the required amount of energy content and/or nutrient content and/or micro nutrient that are required to match the first data with the second data and calculating the number of basic amounts of the selected fortifier that brings the energy content and/or nutrient content and/or micro nutrient content closest to the required energy content and/or nutrient content and/or micro nutrient content.

The software can also have instructions for driving a pumping station to deliver the feed to the infant according to the feed order. This is optional anyway, because this step can be manually implemented by the healthcare professional.

While various aspects of the present disclosure have been described herein, it will be appreciated that variations may be made thereto that are still within the scope of the appended claims.

What is claimed is:

1. A method for administering breastmilk to an infant, comprising:
providing breastmilk;
providing first data indicative of at least one of a group of an energy content, a nutrient content, and a micro nutrient content of the provided breastmilk;
providing second data indicative of the at least one of the group of the energy content, the nutrient content, and the micro nutrient content included in a feed order for the infant to whom the breastmilk is to be administered;
comparing the first data to the second data;
If the first data do not match the second data,
automatically
(i) determining at least one fortifier and a quantity thereof to be added to the breastmilk for matching the first data and the second data;
(ii) introducing the determined quantity of each of the at least one fortifier into the breastmilk; and
delivering the fortified breastmilk to the infant.

2. The method of claim 1, wherein providing first data includes providing average data indicative of the at least one of the group of energy content, nutrient content, and micro nutrient content of the provided breastmilk.

3. The method of claim 1, wherein providing first data includes analyzing the milk and determining at least one of the group of the energy content, nutrient content, and micro nutrient content of the provided breastmilk.

4. The method of claim 1, wherein determining at least one fortifier includes
providing a database of fortifiers, each fortifier in the database being linked to at least one of a group of its energy content, nutrient content, and micro nutrient content;
determining from the comparison the required energy content, nutrient content, and/or micro nutrient content required to match the first data with the second data;
selecting from the database the fortifier linked to the at least one of the group of the energy content, the nutrient content, and the micro nutrient content that corresponds to the required energy content, nutrient content, and/or micro nutrient content.

5. The method of claim 1, wherein determining the quantity of the at least one fortifier includes
each fortifier in the database being linked to a basic amount of that fortifier;
determining from the comparison the required amount of at least one of the group of the energy content, the nutrient content, and the micro nutrient content required to match the first data with the second data;
calculating the number of basic amounts of the selected fortifier that brings the energy content, the nutrient content, and/or the micro nutrient content closest to the required energy content, nutrient content, and/or micro nutrient content.

6. The method of claim 1, wherein providing breastmilk comprises
collecting breastmilk in a collection container;
storing the collection container in a storage device; and
retrieving the collection container from the storage device.

7. The method of claim 1, wherein heating the fortified breastmilk before delivering it to the infant.

8. The method of claim 1, and pasteurizing the breastmilk before delivering it to the infant.

9. A system for administering breastmilk to an infant, comprising:
a collection container;
a fortification station for adding fortifiers into the breastmilk;
a delivery station for delivering the breastmilk to the infant;
a computer system with a processing unit for
receiving first data indicative of at least one of a group of an energy content, a nutrient content, and a micro nutrient content of the provided breastmilk;
receiving second data indicative of the at least one of a group of the energy content, the nutrient content, and the micro nutrient content of a feed order for the infant to whom the breastmilk is to be administered;
comparing the first data with the second data, and
if the first data do not match the second data, automatically determining at least one fortifier and the quantity thereof to be added to the breastmilk for matching the first data and the second data.

10. The system according to claim 9, further comprising reservoirs for the fortifiers with a dispensing unit, the computer system being connected to and driving the dispensing units for introducing the determined quantity of each of the at least one fortifier into the breastmilk.

11. A software for administering breastmilk to an infant, stored on a non-transitory computer readable medium, comprising instructions that, when read by a processing unit of a computer system, cause the processing unit to
receive first data indicative of at least one of a group of an energy content, a nutrient content, and a micro nutrient content of the provided breastmilk;
receive second data indicative of the energy content and/or nutrient content and/or micro nutrient content of a feed order for the infant to whom the breastmilk is to be administered;
compare the first data with the second data;
if the first data do not match the second data, automatically determining at least one fortifier and the quantity thereof to be added to the breastmilk for matching the first data and the second data.

12. The software of claim 11, further comprising instructions for driving a pumping station.

13. The software of claim 11, further comprising a database of fortifiers linking each fortifier in the database to at least one of the group of the energy content, the nutrient content, and the micro nutrient content thereof, and further comprising instructions for
determining from the comparison the required energy content and/or nutrient content and/or micro nutrient that are required to match the first data with the second data
selecting from the database the fortifier linked to the energy content, the nutrient content, and/or the micro nutrient content that corresponds to the required energy content, the nutrient content, and/or the micro nutrient content.

14. The software of claim 11, wherein the database links each fortifier to basic amounts thereof, further comprising instructions for
determining from the comparison the required amount of the energy content, the nutrient content, and/or the micron nutrient content required to match the first data with the second data; and
calculating the number of basic amounts of the selected fortifier that brings the energy content, the nutrient content, and/or the micron nutrient content closest to the required energy content, nutrient content, and/or micron nutrient content.
